(19) United States
(12) Patent Application Publication
(10) Pub. No.: US 2009/0118569 A1
(43) Pub. Date: May 7, 2009

(54) Premixed Counseling and Sound Therapy Matrix For Single Media Players

(76) Inventor: Michael Petroff, (US)

Correspondence Address: TRAN & ASSOCIATES, 6768 MEADOW VISTA CT., SAN JOSE, CA, 95135, US

(21) Appl. No.: 11/934,045

(22) Filed: Nov. 1, 2007

(51) Int. Cl. A61M 21/02; G09B 19/00

(52) U.S. Cl. 600/28; 434/236

(57) ABSTRACT

A matrixed and premixed counseling and sound therapy program format for single media players, which provides, and enables user-selection among, a multiplicity of differing music or other sound therapy tracks each separately premixed with a common, or substantially equivalent or redundant counseling track. The system may be applied to more than one substantially different counseling program each based on a common redundant counseling track. The program format is applicable to a diversity of therapeutic, self-improvement, training and educational programs.

### FIELD OF THE INVENTION

The present invention relates to sound program formats for media players, and in particular to pre-mixed counseling and sound therapy program formats for single media players.

### BACKGROUND OF THE INVENTION

The advantages and effectiveness of recorded counseling relating to various medical, psychological and personal enhancement issues have been widely known for many decades. More recently, music therapy has become a burgeoning health care field in which music and other sounds have been shown to substantially enhance patient tolerance and management of a wide range of physical and psychological disorders. Simultaneous combinations of counseling and music or other sounds can be particularly effective. Prior art premixed recorded combinations of counseling and music or other sounds have been used in various therapeutic, self-improvement, training and educational programs delivered to a user by a single media player. Such combinations have been recorded and stored on single media players in such a manner as to provide non-redundant counseling content, which ostensibly has been sufficient and optimal in terms of information delivery. Therefore it is not surprising that such prior art premixed combinations have not been recorded in such a manner as to provide, and enable users to select one among, a multiplicity of differing music or other sound tracks each separately premixed with a common, or substantially equivalent, and therefore redundant counseling track. Further, once a music or other sound track has been premixed with a counseling track and stored on a single media player, neither such counseling track nor such music or other sound can later be separated or remixed in other combinations using such single media player, and thus the above described user preference relating to multiple music or sound therapy options cannot be addressed by prior art premixed recorded combinations.

### SUMMARY OF THE INVENTION

In one aspect, a method to provide a matrix of premixed counseling and content tracks, configured for single media player includes providing a menu of a plurality of sound therapy tracks comprising a plurality of content tracks each separately premixed with a redundant counseling track; selecting a sound therapy track from the menu; and playing the selected sound therapy track on a single media player.

In another aspect, a system to provide spatially enhanced content sound includes at least one unmodified content track; at least one modified content track derived from an unmodified content track processed by at least one of (i) reverberation, (ii) time delay, (iii) low-pass filtering, (iv) phase shifting, (v) polarity inversion, (vi) attenuation; and at least one spatially enhanced content track comprising at least one of (i) a modified content track, or (ii) an unmodified content track summed with a modified content track derived from a sample of such unmodified content track.

In yet another aspect, a system to provide spatially enhanced counseling sound, configured for a single media player includes at least one unmodified counseling track; at least one modified counseling track derived from an unmodified counseling track processed by at least one of (i) reverberation, (ii) time delay, (iii) low-pass filtering, (iv) phase shifting, (v) polarity inversion, (vi) attenuation; at least one spatially enhanced counseling track comprising at least one of (i) a modified counseling track, (ii) an unmodified counseling track summed with a modified counseling track derived from a sample of such unmodified counseling track.

In yet another aspect, a system provides a matrix of premixed counseling and content tracks, configured for a single media player, includes a menu of at least two groups of counseling tracks, each such group comprising a plurality of content tracks, wherein each content track within a group is separately premixed with a common counseling track, wherein common counseling tracks are substantially different for each group, wherein the common counseling track for each group is redundantly comprised in each counseling track within the corresponding group; selecting a counseling track within a group from the menu; and, playing the selected counseling track using a single media player.

Implementations of the above aspect(s) may include one or more of the following. The modified counseling track can introduce differing spatial qualities to the spatially enhanced counseling tracks. The unmodified counseling track can be summed at varying ratios with the modified counseling track, wherein such varying ratios are controlled in at least one of a recording or playback process to introduce differing spatial expansion to the spatially enhanced counseling track. The unmodified counseling track can be onverted to a monaural sound track at varying monaural mix ratios, wherein such varying mix ratios are controlled in at least one of a recording or playback process to introduce differing spatial convergence to the unmodified counseling track. The player can provide a displayed menu or a non-displayed menu. At least one content track or counseling track can be substituted by a spatially enhanced content track.

In one embodiment, a premixed counseling and content track matrix is configured for single media players (meaning single track players of all types including but not limited to iPods and other MP3 players, CD players, computers, playback programs, and wired and wireless transmission devices such as cell phones and the Internet, in each case, however, such players and devices being limited to those not comprising simultaneous multiple sound track mixing or playback functions that are not frequently provided in consumer versions of such players and devices). Such matrix provides, and enables user-selection among, a multiplicity of differing music or other sound therapy tracks each separately premixed with a common, or substantially equivalent, and therefore redundant, counseling track. Within this context, sound therapy tracks are referred to as “content tracks” each premixed with common counseling track. A matrix of such premixed tracks are derived from a recording process of separately premixing a single, and therefore redundant, counseling track with each one of a plurality of differing content tracks. The resulting matrix of premixed tracks, or “format”, is digitally stored on a single media player and is comprised in a displayed or non-displayed menu of such player, whereby each such premixed track may be separately played by such player and is referred to as a “sound therapy track”. The expression “single media player” refers to a media player capable or playing only a single sound track at any given time, such as an iPod and the like. More specifically, in the above recording and single player storage and playback processes, matrixed tracks M consist of a number “n” of content tracks A, each separately premixed with a common, or substantially equivalent, and therefore redundant counseling track B1, constituting matrix tracks M=(B1A1 . . . BAn). In a similar manner, a plurality of groups of such matrixed tracks may be generated, wherein each group is based on a substantially different counseling track that is common, and therefore redundant, within the corresponding group. Such matrixed tracks and groups of matrixed tracks are derived from a recording process, stored on a single media player, and comprised in at least one displayed or non-displayed menus within such player. In order to provide users with choices, a variety of content tracks can be premixed with a given common counseling track, typically 2<A<100. In the preferred embodiment, content tracks A1, A2 and A3 are each separately premixed with common counseling track B1 and constitute first group “1g” of matrixed tracks M1g=(B1A1, B1A2, B1A3); content tracks A1, A2 and A3 are each separately premixed with common counseling track B2 and constitute second group “2g” of matrixed tracks M2g=(B2A1, B2A2, B2A3); and content tracks A1, A2 and A3 are each separately premixed with common counseling track B3 and constitute third group “3g” of matrixed tracks M3g=(B3A1, B3A2, B3A3), collectively providing matrixed sound therapy tracks Mt=(B1A1, B1A2, B1A3)+(B2A1, B2A2, B2A3)+(B3A1, B3A2, B3A3).

The matrixed tracks of various embodiments of the present invention may be applied to a diversity of therapeutic, self-improvement, training or educational programs, in which at least one of such A content tracks may be substituted by an alternative A track not comprised strictly of music, such as but not limited to voices, tones, noise, binaural beat frequencies, nature sounds, or tinnitus masking sounds such as DTM sounds, wherein such alternative A track may further comprise music components. The matrixed tricks of various embodiments of the present invention may further be applied to a diversity of therapeutic, self-improvement, training or educational programs, in which at least one such common, or substantially equivalent, B counseling track may be substituted an alternative B track not comprised strictly of counseling, such as but not limited to voices, tones, noise, binaural beat frequencies, nature sounds, or tinnitus masking sounds such as DTM sounds, wherein such alternative B track may further comprise music components.

Advantages of embodiments of the system of the present invention may include one or more of the following. The system enables the derivation of a matrix of sound therapy tracks configured to provide, and enables user-selection among, a multiplicity of differing content tracks each separately premixed with a common, or substantially equivalent, redundant counseling track. The system further enables any number of groups of such premixed sound therapy tracks (or matrixed tracks), wherein each such group is based on a substantially different common and redundant counseling track, and wherein such groups of tracks are recorded and later stored in at least one menu in a cost-effective, easy to operate single media player. Such matrixed track format is applicable to a diversity of therapeutic, self-improvement, training and educational programs, wherein at least one of such A music tracks may be substituted by a first alternative track not comprised strictly of music, and such common, or substantially equivalent, B counseling track may be substituted a second alternative track not comprised strictly of counseling. The system enables a health care professional or patient/user to listen to a desired counseling program in combination with any of multiple content material options, such as multiple music therapy material options, in accordance with the patient's/user's needs or preferences, and to do so using commonly available, low cost, non-simultaneous multiple sound track players such as MP3 players, iPods and the like. The resulting premixed counseling and content track matrix format for use with a single media player provides, among other benefits, substantially expanded user-controlled sound therapy content flexibility and practicality, and thereby facilitates enhanced sound therapy efficacy. The system enables users to select one among, a multiplicity of differing music or other sound tracks each separately premixed with a common, or substantially equivalent, and therefore redundant counseling track. Other features of the present invention will become apparent from the following descriptions considered in conjunction with the accompanying drawings. It is to be understood that the drawings are not necessarily drawn proportionately or in scale and, unless otherwise indicated, are merely intended to conceptually illustrate the structures, processes and functions described herein. Designated letters and numbers in each of the following drawings correspond to like designated letters and numbers in the remaining drawings.

### BRIEF DESCRIPTIONS OF THE DRAWINGS

FIG. 1 is a chart of a prior art premixed counseling and content track, which provides non-redundant counseling and constitutes prior art sound therapy.

FIG. 2 is a chart of an example of a premixed matrix of counseling and content tracks configured for a single media player, and which provides redundant counseling in accordance with one embodiment of the present invention.

FIG. 3 is a chart of an exemplary first, second and third group of premixed matrixes of counseling and content tracks configured for a single media player, wherein each such group is based on a substantially different counseling track, and wherein each content track within a group is separately premixed with a common counseling track, and which provides redundant counseling in accordance with the preferred embodiment of the present invention.

FIG. 4 is a block diagram of a prior art process for recording, storing and playing counseling and content tracks.

FIG. 5 is a block diagram of an exemplary process for recording, storing and playing premixed matrix of counseling and content tracks in accordance with specific embodiments of the present invention.

FIG. 6 is a block diagram of an exemplary process for recording, storing and playing multiple groups of premixed matrixes of counseling and content tracks in accordance with various embodiments of the present invention.

FIG. 7 is a block diagram of an exemplary process for generating spatially enhanced content sound in accordance with specific embodiments of the present invention.

FIG. 8 is a block diagram of an exemplary process for converting stereophonic content sound to monaural content sound.

FIG. 9 is a block diagram of an exemplary process for generating spatially enhanced counseling sound in accordance with specific embodiments of the present invention.

FIG. 10 is a block diagram of an exemplary process for converting stereophonic counseling sound to monaural counseling sound.

### DESCRIPTION

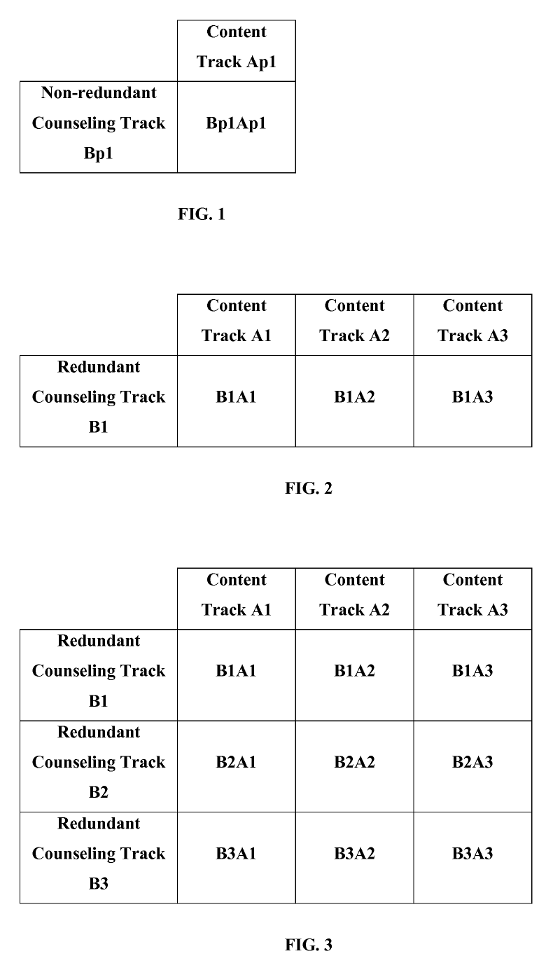

FIG. 1 is a chart of a prior art premixed counseling and content track, which provides non-redundant counseling and constitutes prior art sound therapy, and in which counseling track Bp1 is premixed with content track Ap1 to provide prior art sound therapy track Bp1Ap1.

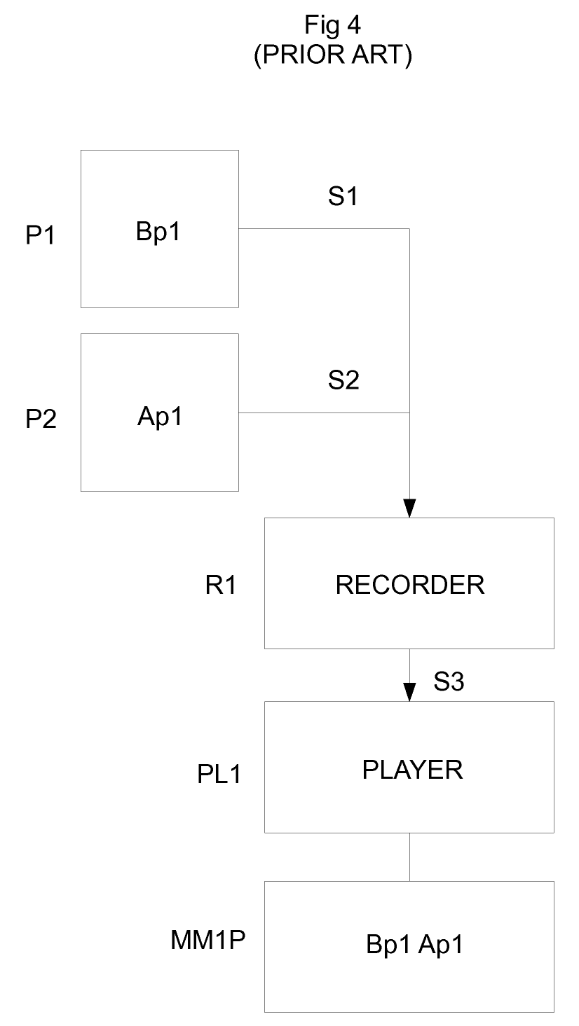

FIG. 2 is a chart of an example of a premixed matrix of counseling and content tracks configured for a single media player, which provide redundant counseling, in which counseling track B1 is separately premixed with each of content tracks A1, A2 and A3 to provide matrix tracks B1A1, B1A2 and B1A3, respectively; counseling track B2 is separately premixed with each of content tracks A1 A2 and A3 to provide matrix tracks B2A1, B2A2 and B2A3, respectively; and counseling track B3 is separately premixed with each of content tracks A1, A2 and A3 to provide matrix tracks B3A1, B3A2 and B3A3, respectively. The example shows premixed matrix tracks M=(B1A1 . . . BAn).

FIG. 3 is a chart of an example showing first, second and third groups of premixed matrixes of counseling and content tracks configured for a single media player, wherein each such group is based on a substantially different counseling track, wherein each content track within a group is separately premixed with a common counseling track, which provides redundant counseling, in which content tracks A1, A2 and A3 are each separately premixed with common counseling track B1 and constitute first group “1g” of matrixed tracks M1g=(B1A1, B1A2, B1A3); content tracks A1, A2 and A3 are each separately premixed with common counseling track B2 and constitute second group 2g of matrixed tracks M2g=(B2A1, B2A2, B2A3), and content tracks A1, A2 and A3 are each separately pre-mixed with common counseling track B3 and constitute third group 3g of matrixed tracks M3g=(B3A1, B3A2, B3A3), collectively providing matrixed tracks Mt=(B1A1, B1A2, B1A3)+(B2A1, B2A2, B2A3)+(B3A1, B3A2, B3A3).

FIG. 4 is a block diagram of a prior art process for recording, storing and playing counseling and content tracks, in which program input P1, consisting of counseling track Bp1, constitutes signal S1 applied to a first input of recorder R1. Program input P2, consisting of content track Ap1, constitutes signal S2 applied to a second input of recorder R1. Recorder R1 internally mixes input signals S1 and S2, stores the resulting sound therapy track Bp1Ap1, and provides corresponding output signal S3 applied as input to player PL1. Player PL1 stores and plays track Bp1Ap1, which is comprised in menu MM1p of PL1.

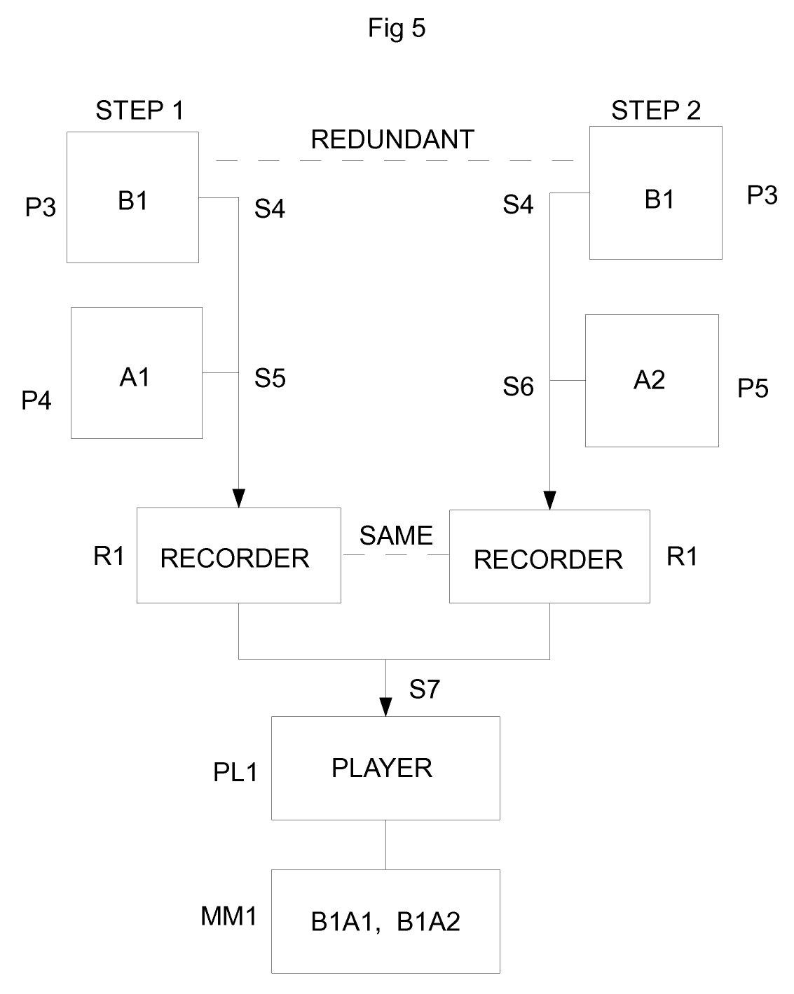

FIG. 5 is a block diagram of an exemplary process for recording, storing and playing premixed matrix of counseling and content tracks in accordance with specific embodiments of the present invention, in which, in a first step, program input P3, consisting of counseling track B1, constitutes signal S4 applied to a first input of recorder R1. Program input P4, consisting of content track A1, constitutes signal S5 applied to a second input of recorder R1. Recorder R1 internally mixes input signals S4 and S5, stores the resulting sound therapy track B1A1, and provides corresponding output signal S7 applied as input to player PL1. Player PL1 stores and plays track B1A1, which is comprised in matrix menu MM1 of PL1. In a second step, program input P3, consisting of counseling track B1, constitutes signal S4 applied to a first input of recorder R1. Program input P5, consisting of content track A2, constitutes signal S6 applied to a second input of recorder R1. Recorder R1 internally mixes input signals S5 and S6, stores the resulting sound therapy track B1A2, and provides corresponding output signal S7 applied as input to player PL1. Player PL1 stores and plays track B1A1 and B1A2, which are comprised in matrix menu MM1 of PL1, and in which counseling therapy is provided redundantly.

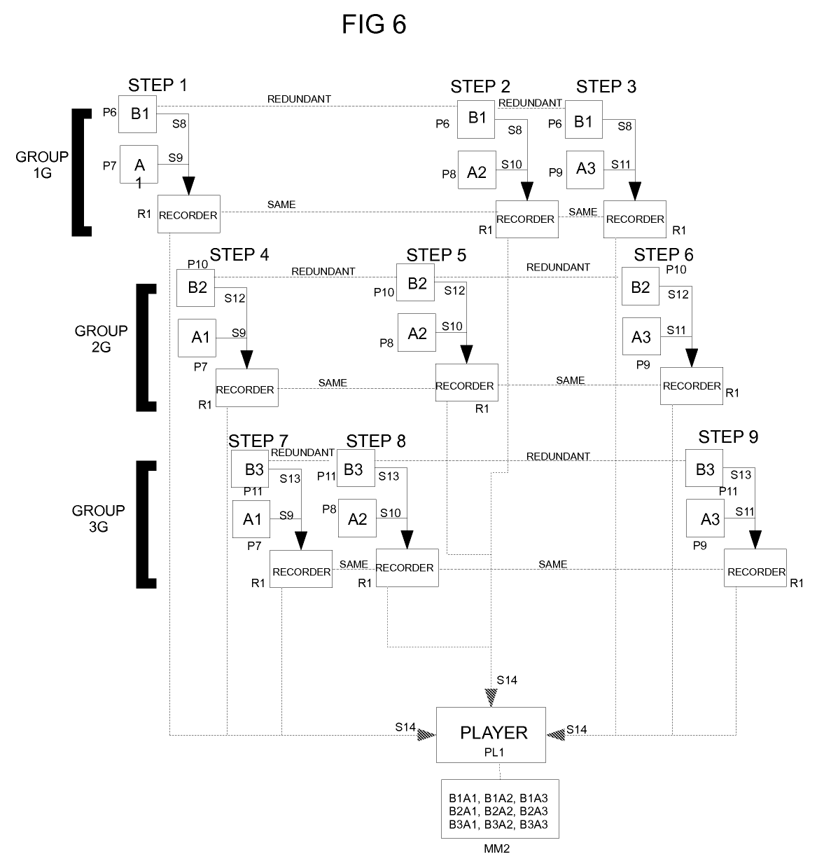

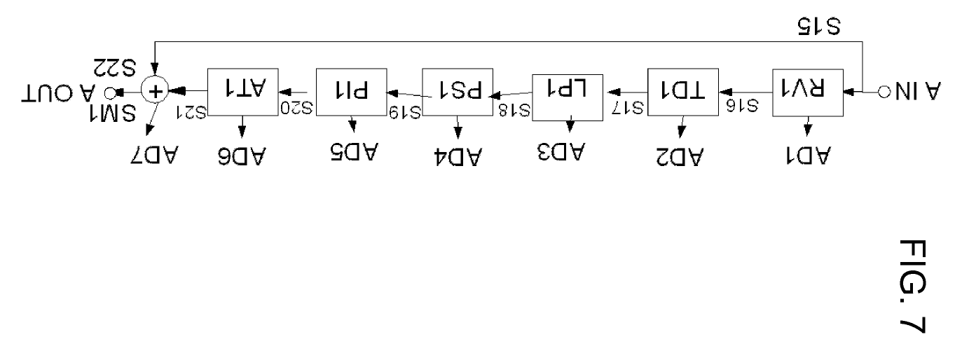

FIG. 6 is a block diagram of an exemplary process for recording, storing and playing multiple groups of premixed matrixes of counseling and content tracks in accordance with specific embodiments of the present invention, in which, in a first step, program input P6, consisting of counseling track B1, constitutes signal S8 applied to a first input of recorder R1. Program input P7, consisting of content track A1, constitutes signal S9 applied to a second input of recorder R1. Recorder R1 internally mixes input signals S8 and S9, stores the resulting sound therapy track B1A1, and provides corresponding output signal S14 applied as input to player PL1. Player PL1 stores and plays track B1A1, which is comprised in matrix menu MM1 of PL1. In a second step, program input P6, consisting of counseling track B1, constitutes signal S8 applied to a first input of recorder R1. Program input P8, consisting of content track A2, constitutes signal S10 applied to a second input of recorder R1. Recorder R1 internally mixes input signals S8 and S10, stores the resulting sound therapy track B1A2, and provides corresponding output signal S14 applied as input to player PL1. Player PL1 stores and plays track B1A2, which is comprised in matrix menu MM1 of PL1. In a third step, program input P6, consisting of counseling track B1, constitutes signal S8 applied to a first input of recorder R1. Program input P9, consisting of content track A3, constitutes signal S11 applied to a second input of recorder R1. Recorder R1 internally mixes input signals S8 and S11, stores the resulting sound therapy track B1A3, and provides corresponding output signal S14 applied as input to player PL1. Player PL1 stores and plays track B1A3, which is comprised in matrix menu MM1 of PL1. Tracks B1A1, B1A2 and B1A3 above constitute a first group 1g of matrixed sound therapy tracks, in which counseling therapy is provided redundantly. In a fourth step, program input P10, consisting of counseling track B2, constitutes signal S12 applied to a first input of recorder R1. Program input P7, consisting of content track A1, constitutes signal S9 applied to a second input of recorder R1. Recorder R1 internally mixes input signals S9 and S12, stores the resulting sound therapy track B2A1, and provides corresponding output signal S14 applied as input to player PL1. Player PL1 stores and plays track B2A1, which is comprised in matrix menu MM1 of PL1. In a fifth step, program input P10, consisting of counseling track B2, constitutes signal S12 applied to a first input of recorder R1. Program input P8, consisting of content track A2, constitutes signal S10 applied to a second input of recorder R1. Recorder R1 internally mixes input signals S10 and S12, stores the resulting sound therapy track B2A2, and provides corresponding output signal S14 applied as input to player PL1. Player PL1 stores and plays track B2A2, which is comprised in matrix menu MM1 of PL1. In a sixth step, program input P10, consisting of counseling track B2, constitutes signal S12 applied to a first input of recorder R1. Program input P9, consisting of content track A3, constitutes signal S11 applied to a second input of recorder R1. Recorder R1 internally mixes input signals S11 and S12, stores the resulting sound therapy track B2A3, and provides corresponding output signal S14 applied as input to player PL1. Player PL1 stores and plays track B2A3, which is comprised in matrix menu MM1 of PL1. Tracks B2A1, B2A2 and B2A3 above constitute a second group 2g of matrixed sound therapy tracks, in which counseling therapy is provided redundantly. In a seventh step, program input P11, consisting of counseling track B3, constitutes signal S13 applied to a first input of recorder R1. Program input P7, consisting of content track A1, constitutes signal S9 applied to a second input of recorder R1. Recorder R1 internally mixes input signals S9 and S13, stores the resulting sound therapy track B3A1, and provides corresponding output signal S14 applied as input to player PL1. Player PL1 stores and plays track B3A1, which is comprised in matrix menu MM1 of PL1. In an eighth step, program input P11, consisting of counseling track B3, constitutes signal S13 applied to a first input of recorder R1. Program input P8, consisting of content track A2, constitutes signal 10 applied to a second input of recorder R1. Recorder R1 internally mixes input signals S10 and S13, stores the resulting sound therapy track B3A2, and provides corresponding output signal S14 applied as input to player PL1. Player PL1 stores and plays track B3A2, which is comprised in matrix menu MM1 of PL1. In a ninth step, program input P11, consisting of counseling track B3, constitutes signal S13 applied to a first input of recorder R1. Program input P9, consisting of content track A3, constitutes signal S11 applied to a second input of recorder R1. Recorder R1 internally mixes input signals S11 and S13, stores the resulting sound therapy track B3A3, and provides corresponding output signal S14 applied as input to player PL1. Player PL1 stores and plays track B3A3, which is comprised in matrix menu MM1 of PL1. Tracks B3A1, B3A2 and B3A3 above constitute a third group 3g of matrixed sound therapy tracks, in which counseling therapy is provided redundantly.

FIG. 7 is a block diagram of an exemplary process for generating spatially enhanced content sound in accordance with specific embodiments of the present invention, in which content source A IN constitutes unmodified content signal S15. S15 is applied as input to reverberation module RV1 having reverberation adjustment AD1. VRI provides output signal S16, which is applied as input to time delay TD1 having time delay adjustment AD2. TD1 provides output signal S17, which is applied as input to low-pass filter LP1 having low-pass filtering adjustment AD3. LP1 provides output signal S18, which is applied as input to phase shifter PS1 having phase shift adjustment AD4. PS1 provides output signal S19, which is applied as input to phase inverter PI1 having phase inversion adjustment (in/out) AD5. PI1 provides output signal S20, which is applied as input to attenuator AT1 having attenuation adjustment AD6. AT1 provides output signal S21, which constitutes a modified content signal, and which is applied to a first input of summer SM1 having summing ratio adjustment AD7. Unmodified content signal S15 is applied to a second input of summer SM1. SM1 provides output signal S22, which constitutes a spatially enhanced content signal A′.

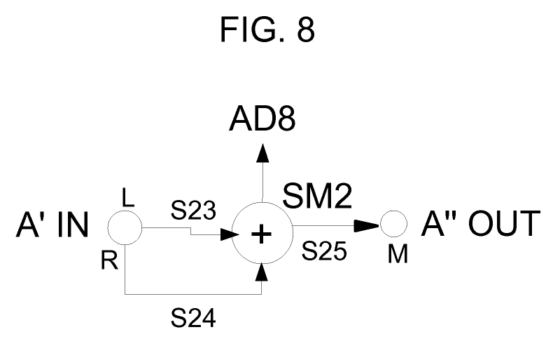

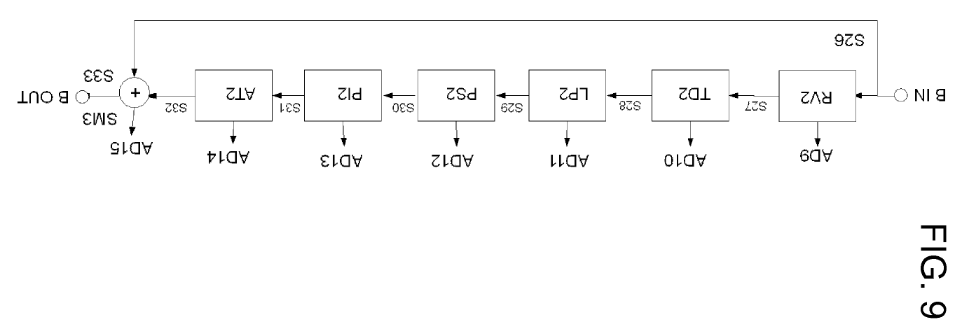

FIG. 8 is a block diagram of an exemplary process for converting stereophonic content sound to monaural content sound, in which spatially enhanced content signal A′ comprises left (L) and a right (R) stereophonic signal S23 and S24, respectively, applied to a first and second input of summer SM2, respectively. SM2 has monaural mixing adjustment AD8 and provides monaural (M) output signal S25. S25 constitutes a further modified spatially enhanced content output signal A″.

FIG. 9 is a block diagram of an exemplary process for generating spatially enhanced counseling sound in accordance with specific embodiments of the present invention, in which counseling source B IN constitutes unmodified content signal S26. S26 is applied as input to reverberation module RV2 having reverberation adjustment AD9. VR2 provides output signal S27, which is applied as input to time delay TD2 having time delay adjustment AD10. TD2 provides output signal S28, which is applied as input to low-pass filter LP2 having low-pass filtering adjustment AD11. LP2 provides output signal S29, which is applied as input to phase shifter PS2 having phase shift adjustment AD12. PS2 provides output signal S30, which is applied as input to phase inverter PI2 having phase inversion adjustment (in/out) AD13. PI2 provides output signal S31, which is applied as input to attenuator AT2 having attenuation adjustment AD14. AT2 provides output signal S32, which constitutes a modified counseling signal, and which is applied to a first input of summer SM3 having summing ratio adjustment AD15. Unmodified counseling signal S25 is applied to a second input of summer SM3. SM3 provides output signal S33, which constitutes a spatially enhanced counseling signal B′.

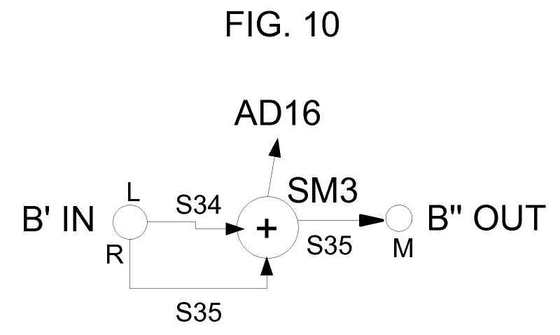

FIG. 10 is a block diagram of an exemplary process for converting stereophonic counseling sound to monaural counseling sound, in which spatially enhanced counseling signal B′ comprises left (L) and a right (R) stereophonic signal S34 and S35, respectively, applied to a first and second input of summer SM3, respectively. SM3 has monaural mixing adjustment AD16 and provides monaural (M) output signal S36. S36 constitutes a further modified spatially enhanced counseling output signal B″.

The techniques described herein may be implemented in hardware or software, or a combination of the two. Preferably, the techniques for converting the counseling tracks, and content tracks (such as music therapy), into a matrix of sound therapy content that can be played by a single media player are implemented by computer recording programs. Such techniques may also be implemented in a more manual fashion using audio mixers whereby a matrix of such counseling tracks and content tracks are premixed and recorded. Each such premixed counseling and content track can be stored on a storage medium or device (e.g., flash memory, CD-ROM, hard disk or magnetic diskette) that is readable by a general or special purpose programmable microcontroller, microprocessor or digital signal processor.

The system enables sound therapy matrixes in which counseling, music and/or other sound formats substantially enhance patient tolerance and management of a wide range of physical and psychological disorders, such as stress and stress-related disorders, cancer and other life-threatening conditions, pre-natal care and birthing management, fibromyalgia, chronic fatigue syndrome, chronic or acute pain, addictions issues, depression or anxiety, relationship challenges, hospice care needs, grief and loss, and trauma management, among others.

When used to deliver music therapy, the system enables an enhancement of the positive effects of music therapy on patients. Clinically, patients are more likely to perceive that they received a higher quality of care. The use of such enhanced music therapy preoperatively can be an excellent noninvasive technique, can reduce hospital stay time, and can offer patients additional comfort in stressful clinical environments. Music therapy has been clinically shown to reduce anxiety, lower blood pressure, heart and breathing rates, and relax muscular tension.

While the foregoing discusses matrixed combinations of counseling tracks and content tracks based on music therapy or other sound formats, the present invention is not so limited. For example, learning to play a musical instrument is another form of music therapy wherein the principles of the present invention may be applied. The user can choose from a matrixed menu a particular form of music that reflects the mood that he or she is currently feeling, or would like to feel. The system can then play, in a simple to operate manner, the corresponding premixed counseling and selected music tracks. In this example the counseling track would consist of an instructional track that teaches one how to play such instrument, to arrive at an overall sound therapy program that enhances the quality of life for the user.

The above-described embodiments of the present invention are merely meant to be illustrative and not limiting. Various changes and modifications may be made without departing from the invention in its broader aspects. The appended claims encompass such changes and modifications within the spirit and scope of the invention.

What is claimed is:

1. A method to provide a matrix of premixed counseling and content tracks, configured for single media player, comprising:
a. providing a menu of a plurality of sound therapy tracks comprising a plurality of content tracks each separately premixed with a redundant counseling track;
b. selecting a sound therapy track from the menu; and
c. playing the selected sound therapy track on a single media player.

2. The method of claim 1, wherein at least one of the content tracks comprises one of: (i) music, (ii) voices, (iii) tones, (iv) noise, (v) binaural beat frequencies, (vi) nature sounds, (vii) tinnitus therapy sounds, (viii) DTM tinnitus mitigation sounds.

3. The method of claim 1, wherein at least one of the counseling tracks comprises one of: (i) music, (ii) voices, (iii) tones, (iv) noise, (v) binaural beat frequencies, (vi) nature sounds, (vii) tinnitus therapy sounds, (viii) DTM tinnitus mitigation sounds, (ix) psychological advice, (x) medical advice, (xi) training, (xii) education, (xiii) entertainment.

4. The system of claim 1, wherein the player provides one of: a displayed menu, a non-displayed menu.

5. A system to provide spatially enhanced content sound, comprising:
a. at least one unmodified content track;
b. at least one modified content track derived from an unmodified content track processed by at least one of (i) reverberation, (ii) time delay, (iii) low-pass filtering, (iv) phase shifting, (v) polarity inversion, (vi) attenuation; and
c. at least one spatially enhanced content track comprising at least one of (i) a modified content track, or (ii) an unmodified content track summed with a modified content track derived from a sample of such unmodified content track.

6. The system of claim 5, wherein the modified content track is adjusted in at least one of a recording or playback process to introduce differing spatial qualities to the spatially enhanced content tracks.

7. The system of claim 5, wherein the unmodified content track is summed at varying ratios with the modified content track, and wherein the varying ratios are controlled in at least one of a recording or playback process to introduce differing spatial expansion to the spatially enhanced content track.

8. The system of claim 5, in which the unmodified content track is converted to a monaural sound track at varying monaural mix ratios, wherein such varying mix ratios are controlled to introduce differing spatial convergence to the unmodified content track.

9. The system of claim 5, wherein the player provides one of: a displayed menu, a non-displayed menu.

10. A system to provide spatially enhanced counseling sound, configured for a single media player, comprising:
a. at least one unmodified counseling track;
b. at least one modified counseling track derived from an unmodified counseling track processed by at least one of (i) reverberation, (ii) time delay, (iii) low-pass filtering, (iv) phase shifting, (v) polarity inversion, (vi) attenuation;
c. at least one spatially enhanced counseling track comprising at least one of (i) a modified counseling track, (ii) an unmodified counseling track summed with a modified counseling track derived from a sample of such unmodified counseling track;

11. The system of claim 10, wherein the modified counseling track introduces differing spatial qualities to the spatially enhanced counseling tracks.

12. The system of claim 10, wherein the unmodified counseling track is summed at varying ratios with the modified counseling track, wherein such varying ratios are controlled in at least one of a recording or playback process to introduce differing spatial expansion to the spatially enhanced counseling track.

13. The system of claim 10, wherein the unmodified counseling track is converted to a monaural sound track at varying monaural mix ratios, wherein such varying mix ratios are controlled in at least one of a recording or playback process to introduce differing spatial convergence to the unmodified counseling track.

14. The system of claim 10, wherein the player provides one of: a displayed menu, a non-displayed menu.

15. A system to provide a matrix of premixed counseling and content tracks, configured for a single media player, the system comprising:
a. a menu of at least two groups of counseling tracks, each such group comprising a plurality of content tracks, wherein each content track within a group is separately premixed with a common counseling track, wherein common counseling tracks are substantially different for each group, whereby the common counseling track for each group is redundantly comprised in each counseling track within the corresponding group;
b. selecting a counseling track within a group from the menu; and,
c. playing the selected counseling track using a single media player.

16. The system of claim 15, in which at least one content track is substituted by a spatially enhanced content track.

17. The system of claim 15, in which at least one counseling track is substituted by a spatially enhanced counseling track.

18. The system of claim 15, wherein the player provides one of: a displayed menu, a non-displayed menu.
